(19) United States
(12) Patent Application Publication
(10) Pub. No.: US 2009/0043184 A1
(43) Pub. Date: Feb. 12, 2009

(54) CATHETER AND PROBE FOR MEASURING ANALYTES OR OTHER PARAMETERS

(75) Inventors: Todd Fjield, Laguna Hills, CA (US); Michael J. Higgins, Huntington Beach, CA (US)

Correspondence Address: EDWARDS LIFESCIENCES CORPORATION, LEGAL DEPARTMENT, ONE EDWARDS WAY, IRVINE, CA, 92614, US

(73) Assignee: Edwards Lifesciences Corporation, Irvine, CA (US)

(21) Appl. No.: 12/187,943

(22) Filed: Aug. 7, 2008

Related U.S. Application Data
(60) Provisional application No. 60/954,742, filed on Aug. 8, 2007.

(51) Int. Cl. A61B 5/145

(52) U.S. Cl. 600/366

(57) ABSTRACT

A medical sensing system including an access tube with a fluid infusate lumen and a sensing probe that can be passed through a second lumen in the access tube and extended into the vasculature for sensing a blood parameter. The sensing probe has a sensor on a distal end thereof and a shape that permits it to extend from the access tube such that the sensor is displaced laterally from the access tube, and out of the infusate flow. If the sensing probe passes through the lumen opening at the distal tip of the catheter, the distal end of the probe bends or otherwise deflects laterally so that the sensor is displaced laterally from the infusate flow path. Alternatively, the probe may exit a side port of the access tube upstream of an infusate side port. The deflectable probe is particularly useful for sensing glucose in blood through any number of conventional, off-the-shelf catheters.

### CROSS-REFERENCE TO RELATED APPLICATIONS

This application claims priority from U.S. Provisional Patent Application No. 60/954,742, filed Aug. 8, 2007, which is also hereby incorporated herein by reference.

### FIELD OF THE INVENTION

The present invention relates to a medical probe or catheter system including a probe for measuring analytes such as glucose or other parameters.

### BACKGROUND OF THE INVENTION

In a hospital setting there is always the need to monitor patient health through the evaluation of blood chemistry profile. It is often necessary or desirable to measure the presence and/or concentration of blood constituents, such as blood gases, hydrogen ions other electrolytes, glucose, red blood cells and the like. This can be accomplished in an extracorporeal blood loop or continuously and in real time in a patient undergoing surgery or intensive care by utilizing a catheter and a probe. The probe includes one or more sensors which are responsive to the constituent or compositional parameter of interest to provide a signal related to such constituent. One such system is shown in Maxwell U.S. Pat. No. 4,830,013, where in one embodiment a sensor is positioned within the lumen of the catheter and adjacent the distal opening of the catheter. In a blood gas measurement system shown in Nestor, et al., U.S. Pat. No. 4,900,933, a dual-lumen catheter is provided with one lumen being used for fluid infusion and the other lumen being used for the probe. In the Nestor, et al. construction, the probe and the sensor of the probe are located outside the lumen distally of the distal end of the lumen.

Despite many existing systems, there remains a need for a blood parameter sensing system that is more reliable and accurate.

### SUMMARY OF THE INVENTION

In one aspect, the present invention provides a medical system for sensing a parameter of blood. For example, the system can be used to measure an analyte, such as glucose, or a physical property of blood such as oxygenation. Desirably, the system includes an access tube such as a catheter and a sensing probe extendable therefrom. The access tube includes at least one lumen for the introduction of infusates into the vasculature. The sensing probe of the present invention may project from a port in the access tube that is upstream of an infusate port, or may be placed through a distal lumen but be configured to assume a shape such that a sensor thereon resides outside the infusate fluid path.

In an embodiment, the present invention provides a medical sensing system having a vascular access tube, which includes a distal portion configured for insertion into a vasculature, a lumen located within said vascular access tube, an infusate port located at said distal portion of said vascular access tube configured for emitting into the vasculature an infusate input into said lumen during use, and a sensing probe located in said vascular access tube, said sensing probe comprising a sensor located on a distal portion of said sensing probe, wherein said distal portion of said sensing probe is sized to pass through an access port located at said distal portion of said vascular access tube. At least one of said distal portions of said vascular access tube and said sensing probe are configured to substantially eliminate communication between said sensor and infusate emitted from said infusate port of said vascular access tube during use. In an embodiment, said distal portion of said sensing probe is configured for extending from said access port in said vascular access tube so as to orient the sensor at a location outside of a stream of infusate emitted from said infusate port during use.

In an embodiment, said infusate port and said access port are the same port in said vascular access tube. In another embodiment, said vascular access tube is configured such that said infusate port is located more proximal to said distal end of said vascular access tube than said access port. In yet another embodiment, said vascular access tube is configured such that said access port is located more proximal to said distal end of said vascular access tube than said infusate port.

In an embodiment, said distal portion of said sensing probe configured for extending from said access port in said vascular access tube comprises a bend configured to orient the sensor at a location outside of a stream of infusate from said infusate port during use. As an example, said bend in said distal portion of said sensing probe creates a curl in the distal portion of said sensing probe, where the curl is configured to orient said sensor upstream of said access port and along a side of said vascular access tube. As another example, said distal portion of said sensing probe configured for extending from said access port in said vascular access tube comprises first and second bends configured to orient the sensor at a desired location in the vasculature relative to said infusate port. As yet another example, said distal portion of said sensing probe configured to extend from said access port in said vascular access tube comprises a bend portion spaced apart from a distal end of said sensing probe, where the bend portion is configured to orient said sensor at a location upstream of said infusate port during use.

In one aspect, the present invention provides a method of using a medical sensing device for sensing a blood parameter. In an embodiment, the method includes providing a vascular access tube including a fluid infusate lumen and associated fluid infusate port in a sidewall thereof, the access tube further including a second lumen leading to a second port in the access tube; providing a sensing probe having a sensor on a distal end thereof, the sensing probe being sized to pass through the second lumen and having a shape at its distal end that permits the distal end to extend from the second port such that the sensor is displaced laterally from the access tube; introducing the vascular access tube into the vasculature of a patient; passing the sensing probe through the second lumen of the access tube until the distal end extends from the second port; and sensing a blood parameter with the sensor while infusing fluid through the fluid infusate lumen and fluid infusate port into the vasculature.

A further understanding of the nature and application of the present invention are set forth in the following description and claims, particularly when considered in conjunction with the accompanying drawings in which like parts bear like reference numerals.

### BRIEF DESCRIPTION OF THE DRAWINGS

Features of the present invention will become appreciated as the same become better understood with reference to the specification, claims, and appended drawings wherein:

FIG. 1 is an elevational view of a sensing system within a blood vessel having a sensing probe extending linearly from a distal end of a catheter and illustrating a proximal infusion of fluid from the catheter;

FIG. 2 is an elevational view of a sensing system of one embodiment of the present invention within a blood vessel having a sensing probe extending in a non-linear path from a distal end of a catheter and illustrating a proximal infusion of fluid from the catheter;

FIG. 3 is an elevational view of a sensing system of an embodiment of the present invention having an alternative sensing probe extending in a non-linear path from a distal end of a catheter and illustrating a proximal infusion of fluid from the catheter;

FIG. 4 is an elevational view of a sensing system of an embodiment of the present invention having a sensing probe extending from a side port of a catheter and illustrating a distal infusion of fluid from another side port of the catheter; and

FIG. 5 is an elevational view of a sensing system of an embodiment of the present invention having an alternative sensing probe extending from a side port of a catheter and illustrating a distal infusion of fluid from another side port of the catheter.

### DETAILED DESCRIPTION OF THE PREFERRED EMBODIMENTS

The present invention provides a medical sensing system including a sensing probe deliverable through an access tube or catheter. The sensing probe has a sensor on the distal end thereof that can measure glucose or other analytes in the blood. The sensor could also be of a type that measures blood oxygenation, for example for SvO₂ or ScvO₂ monitoring. In a broad sense, a sensing probe is an elongated, flexible, relatively thin medical device that can be passed through a lumen of an access tube, such as a catheter lumen, and which has a sensor disposed on a distal end thereof.

The term analyte as used herein is a broad term, and is to be given its ordinary and customary meaning to a person of ordinary skill in the art (and is not to be limited to a special or customized meaning), and refers without limitation to a substance or chemical constituent in a biological fluid (for example, blood, interstitial fluid, cerebral spinal fluid, lymph fluid or urine) that can be analyzed. Analytes can include naturally occurring substances, artificial substances, metabolites, and/or reaction products. In one desirable embodiment, the analyte for measurement by the sensing devices and methods is glucose. However, other analytes are contemplated as well, including but not limited to acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoyleogonine (cocaine); biotinidase; biopterin; c-reactive protein; carnitine; carnosinase; CD4; ceruloplasmin; chenodeoxycholic acid; chloroquine; cholesterol; cholinesterase; conjugated 1-.beta.hydroxycholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, glucose-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, sexual differentiation, 21-deoxycortisol; desbutylhalofantrine; dihydropteridine reductase; diptheria/tetanus antitoxin; erythrocyte arginase; erythrocyte protoporphyrin; esterase D; fatty acids/acylglycines; free .beta.-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1, .beta.); lysozyme; mefloquine; netilmicin; phenobarbitone; phenytoin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin. Salts, sugar, protein, fat, vitamins, and hormones naturally occurring in blood or interstitial fluids can also constitute analytes in certain embodiments. The analyte can be naturally present in the biological fluid, for example, a metabolic product a hormone, an antigen, an antibody, and the like. Alternatively, the analyte can be introduced into the body, for example, a contrast agent for imaging, a radioisotope, a chemical agent, a fluorocarbon-based synthetic blood, or a drug or pharmaceutical composition, including but not limited to insulin; ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbituates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-dihydroxyphenylacetic acid (DOPAC), homovanillic acid (HVA), 5-hydroxytryptamine (5HT), histamine, Advanced Glycation End Products (AGEs) and 5-hydroxyindoleacetic acid (FHIAA).

The terms sensor and sensing system as used herein are broad terms, and are to be given their ordinary and customary meaning to a person of ordinary skill in the art (and are not to be limited to a special or customized meaning), and refer without limitation to the component or region of a device by which an analyte can be quantified.

The preferred embodiments relate to the use of an analyte sensor that measures a concentration of analyte of interest or a substance indicative of the concentration or presence of the analyte. The analyte sensor can use any method of analyte-sensing, including enzymatic, chemical, physical, electrochemical, spectrophotometric, polarimetric, calorimetric, radiometric, or the like. In addition to analytes, sensors of the present invention may be used to measure blood oxygenation or other parameters of interest. For example, an optical probe may be used for continuous SvO₂ or ScvO₂ monitoring.

The sensors of the present invention may use any method to provide an output signal indicative of the concentration of the analyte or other parameter of interest. The output signal is typically a raw signal that is used to provide a useful value of the parameter of interest to a user, such as a patient or physician, who can be using the device. Accordingly appropriate smoothing, calibration, and evaluation methods can be applied to the raw signal and/or system as a whole to provide relevant and acceptable estimated analyte data to the user.

In one particular embodiment, the methods and devices of preferred embodiments can be employed in a continuous glucose sensor that measures a concentration of glucose or a substance indicative of a concentration or a presence of glucose. The glucose sensor can use any method of glucose-measurement, including calorimetric, enzymatic, chemical, physical, electrochemical, spectrophotometric, polarimetric, calorimetric, radiometric, or the like. In alternative embodiments, the sensor can be any sensor capable of determining the level of an analyte in the body, for example oxygen, lactase, hormones, cholesterol, medicaments, viruses, or the like.

The glucose sensor uses any method to provide an output signal indicative of the concentration of the glucose. The output signal is typically a raw data stream that is used to provide a value indicative of the measured glucose concentration to a patient or doctor, for example.

Central venous (CV) catheters are used primarily to gain access to the venous vasculature for fluid infusion, blood sampling and central venous pressure monitoring. Central venous catheters are relatively long tubular devices which have tapered distal tips that reside in the central circulation, typically in the superior vena cava. There are various types of CV catheters, typically for short term usage. Single-lumen or double-lumen catheters are often inserted for intermittent or continuous infusion of medication of fluid. Multiple lumen catheters have been developed which allow simultaneous introduction of two or more liquids into the vein through a single venous access site. The central venous pressure catheter is a common type of multiple lumen catheter that allows the simultaneous introduction and withdrawal of fluids, as well as the capacity to monitoring blood pressure and other vital parameters.

Swan-Ganz catheters can provide continuous cardiac output (CCO), continuous SvO₂ monitoring, and continuous and the diastolic volume monitoring, plus all of the traditional functions of pulmonary artery catheters. CCO can be measured using the technique of thermodilution, which involves induction at one point of the circulation of a known change in the intravascular heat content of flowing blood and detection of the resultant change in temperature at a point downstream. In particular, using a Swan-Ganz catheter, a known amount of solution with a temperature cooler than blood is injected rapidly into the proximal (upstream) injectate lumen of the catheter. This bolus of solution mixes with the surrounding blood and the temperature is measured downstream in the pulmonary artery by a thermistor bead embedded in the distal end of the catheter. The resultant change in temperature is then plotted on a time-temperature curve.

In addition to various catheters, peripheral lines can be used to sense properties of the bloodstream. A peripheral line refers to a tube that functions much like a catheter in that is inserted into the vasculature and is flexible and has one or more lumens, but which is left in place much longer than a traditional catheter. For example, intravenous lines are typically installed in a patient's arm in the operating room or ICU and remain in place during the procedure and sometimes for a long time afterward. Some of these peripheral lines are suitable for passing other medical instruments therethrough, such as sensing probes.

The preceding descriptions of catheters and sensing methods are used to illustrate the various systems in which the sensing probe of the present invention may be incorporated according to one or more embodiments of the present invention. That is, the probe of present invention of some embodiments can be used in CV catheters, peripheral lines, Swan-Ganz catheters, or various other catheters with lumens. Each of these devices through which an elongated sensing probe can be inserted to access the vasculature possesses at least an elongated tube having one or more lumens.

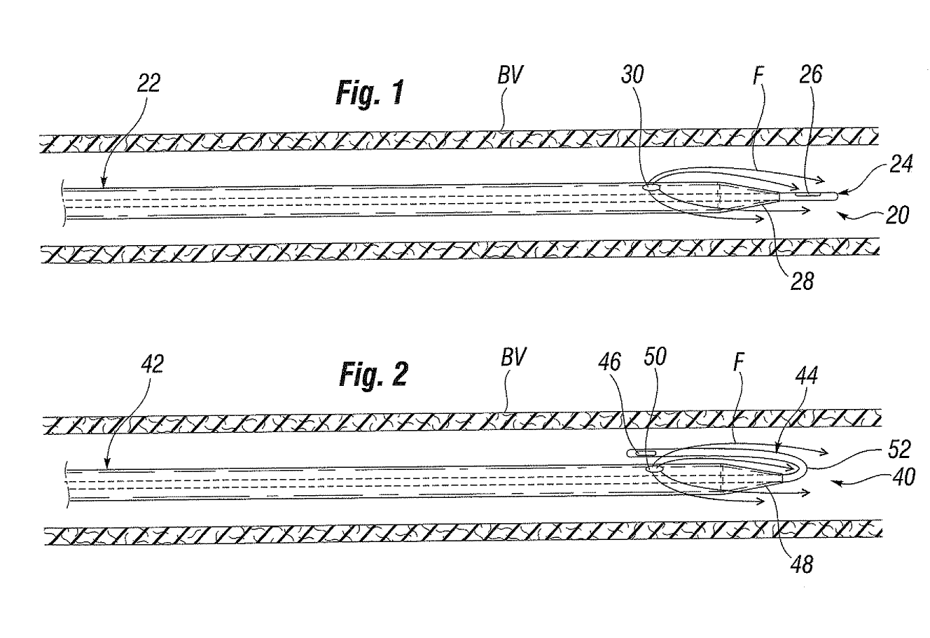

There are several problems with measurements taken with a straight probe through conventional vascular access tubes. For example, measurement of pure blood is often impeded or diluted by the presence of infusates. FIG. 1 illustrates a sensing system 20 comprising an elongated vascular access tube 22 with a sensing probe 24 linearly slidable through a central lumen therein. A distal end of the sensing probe 24 including a sensor 26 may be extended distally from a tapered distal tip 28 of the tube 22. The vascular access tube 22 may be, for example, a conventional CV catheter having a soft, tapered distal tip 28. In the illustrated system 20, the probe 24 extends through a central lumen of the tube 22, such as a guide wire lumen, and projects straight out from the distal tip 28. Often, a side port 30 opens in the wall of the tube 22 at a location that is proximal from the distal tip 28. For example, a conventional CV catheter has at least one and sometimes two such side ports. Fluid infusates such as medications are regularly dispensed through the side port 30, such as indicated by the flow arrows F. As a consequence, the infusate washes over the sensor 26 on the probe 24. The configuration of the system 20, although reliable in some instances, may produce inaccurate readings of blood parameters in the presence of the infusate.

FIG. 2 illustrates one embodiment of a sensing system 40 of the present invention which addresses the drawbacks of sensing systems similar to that shown in FIG. 1. The system 40 also includes an elongated vascular access tube 42, such as a catheter, having a central lumen for receiving a sensing probe 44. The access tube 42 may take the form of any shape known to those having ordinary skill in the art. For example, the access tube 42 may have a circular, ovular, or square cross-section. The sensing probe 44 includes a sensor 46 on the distal end thereof which may be projected distally from a tapered tip 48 of the access tube 42. Also as in FIG. 1, the access tube 42 includes a side port 50 for egress of a fluid infusate F. It should be appreciated that the fluid infusate F may exit via the tapered tip 48. For example, while not shown, the access tube 42 may be configured with two lumens that open to the tapered tip 48. One of the lumens may be configured to receive the sensing probe 44 and another lumen may be configured to receive the fluid infusate F. The lumens may be arranged side-by-side or one inside of the other. It should also be appreciated that the fluid infusate F and the sensing probe 44 may exit the same side port.

Instead of the straight sensing probe 24 of FIG. 1, the system 40 of this embodiment includes a sensing probe 44 that deflects or bends laterally once released from the constraints of the lumen of the access tube 42. For instance, a distal segment of the probe 44 having the sensor 46 thereon is shown extending in a proximal direction due to an approximately 180° bend 52 in the probe. In the illustrated embodiment, the probe 44 projects distally a predetermined distance, for example 2.54 cm (1 inch), from the tapered tip 48 before the bend 52, and then extends in a proximal direction approximately twice that distance (5.08 cm, 2 inches) so that the sensor 46 is laterally spaced from the access tube 42 and preferably located proximal to the side port 50. The sensor 46 is displaced laterally from the access tube 42, and from the axial flow path immediately surrounding the tube, and desirably proximal to the side port 50, such that the sensor 46 is not in the infusate flow F. That is, the sensor 46 remains in the bloodstream and is unaffected by any infusion of fluids through the side port 50.

An aspect of the system 40 of FIG. 2 is to displace the sensor 46 on the probe 44 out of the infusate flow stream F. Of course, there are a number of ways to accomplish this, in addition to lateral displacement of the sensor 46 as illustrated. For example, instead of a 180° bend 52 in the probe 44, the bend 52 could be only 90° such that the sensor 46 projects toward the wall of the blood vessel BV. However, a 90° side bend creates a risk of irritation or trauma to the blood vessel BV by the tip of the probe 44, and therefore to mitigate such trauma the sensor 46 may be positioned just short of the probe tip which is made of extremely soft material. In a still further alternative, the bend 52 may be positioned along the probe 44 with the sensor 46 located proximally upstream of any of the side ports 50. This arrangement thus ensures that infusate will not contaminate the blood management. In addition, extending the proximally directed probe upstream of any of the side ports obviates the need for lateral displacement, such that the sensor 46 can even reside against the access tube 42.

It should be understood that there are a number of ways to create a bend 52 in the probe 44, including both passive and active means. The simplest way to form the bend 52 is to make the probe at least partly from a resilient material that is elastically-biased to form the bend 52. For example, a segment of the probe 44 at the desired location of the bend 52 may be formed of Nitinol and have a relaxed configuration defined by the formed bend. Because certain forms of Nitinol are super-elastic, the probe 44 can be straightened out and passed through the catheter lumen without plastic deformation. Alternatively, the probe 44 may be molded of a polymer material with a small segment of Nitinol wire embedded therein. On the other hand, the probe 44 may be actively deflected by providing a thin actuation wire (not shown) extending therethrough that pulls on the distal tip thereof. By providing a segment of weakness at the location of the bend 52, the distal tip of the probe 44 can be deflected laterally by pulling on the wire. Another technique which is a hybrid of passive and active is to provide a bend segment of material that changes shaped upon a temperature change. For example, certain forms of Nitinol are subject to temperature-induced crystalline phase changes which can be designed to form the bend 52 upon exposure to the bloodstream.

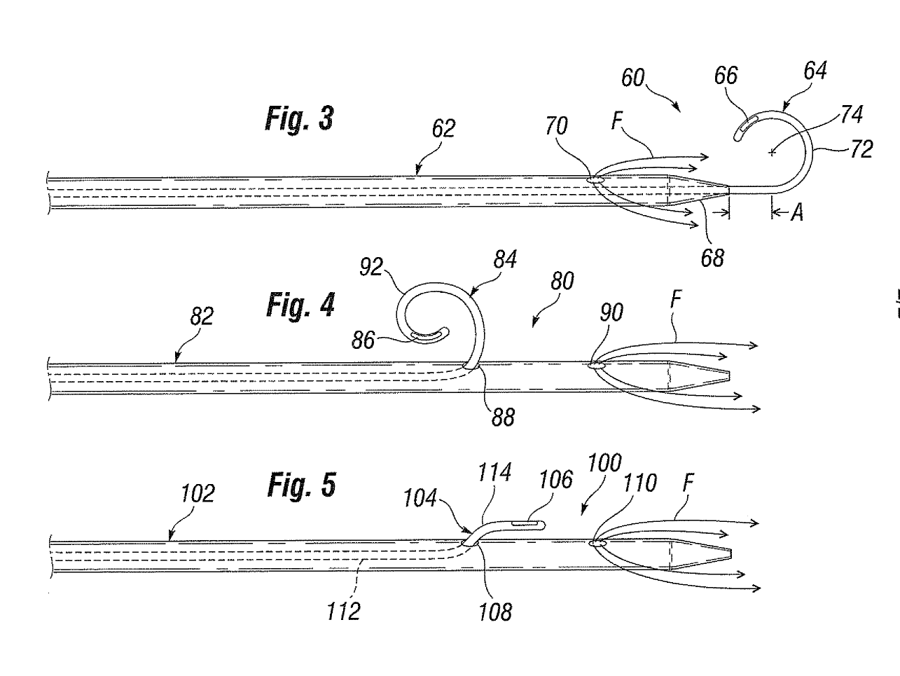

The system 40 illustrated in FIG. 2 shows a relatively sharp bend 52 leading to a somewhat elongated linear distal segment in the probe 44 terminating in the sensor 46. A similar system 60 having an alternative probe is seen in FIG. 3. The system 60 includes an access tube 62 again having a sensing probe 64 linearly slidable through a central lumen of the access tube 62. A distal end of the sensing probe 64 includes a sensor 66 that projects out of a tapered tip 68 of the access tube 62. Also as in the previous embodiment, a proximal side port 70 releases infusate F. The probe 64 forms a substantially circular bend 72 centered about a point 74 as it exits the tapered tip 68. The bend 72 extends between approximately 180-270° so that the distal end of the probe 64 and sensor 66 project in a proximal direction. In addition, the diameter of the bend 72 is such that the sensor 66 is displaced laterally from the exterior of the tube 62 and is therefore out of the main infusate flow F. Ideally, the clinician extends the probe 64 a distance A beyond that which would be necessary to form the complete bend 72 so as to provide some slack for the relative catheter/probe movement as the catheter flexes.

The foregoing describes a probe tip that includes an approximately 180° sharp bend to form a J-shape, and another that bends between 180-270° to form a partial circle or crook shape. However, any such deformation of the distal tip of the sensing probe from linear could be used to encourage the probe to deflect laterally or exit a side port of the access tube.

In each of the examples described herein, the deflections or curves provided in the probes are such as to result in minimal trauma to the surrounding blood vessel. That is, the probes possess atraumatic tips that prevent their distal end points from being directed toward the vessel wall. This helps ensure minimal damage to the blood vessel, and also improves the accuracy of measurement depending on the type and location of the sensor thereon. For example, the sensor may be provided in the distal-most tip of the probe which would be occluded if the tip were projected directly into the vessel wall.

Another solution to problems associated with probe-within-catheter sensing systems is to direct the probe from one of the sides ports of the catheter. Standard CV and other catheters, however, are often not well designed for a probe exiting the side port without some difficulty. To overcome this issue, the probe can be designed with a slight curvature at its distal end to encourage it to exit the side port without getting caught on the edge of the side port.

For instance, FIG. 4 illustrates a sensing system 80 comprising an elongated access tube 82 and a sensing probe 84 having a distal sensor 86 thereon. The illustrated access tube 82, which may be a conventional CV catheter, includes multiple lumens, one each of which lead to a proximal side port 88 and a distal side port 90. While the distal side port 90 may be used to infuse fluid F, the probe 84 extends through the lumen associated with the proximal side port 88 and projects therefrom. To do so, the probe 84 forms a bend or deflection 92 adjacent its distal end, thus causing the distal segment to curl in a proximal direction from the side port 88. The proximal end of the probe 84 could be provided with a marker (not shown) for rotational orientation to ensure that the bend 92 points toward the side port 88. Furthermore, the probe 84 desirably possesses a minimum torque strength so that it can be twisted if necessary until the distal tip thereof exits the side port 88. By projecting the probe 84 from the proximal side port 88, it remains at all times proximally located with respect to the distal side port 90 and infusate flow F, at least when the access tube 82 advances in an antegrade manner. When the access tube 82 advances in an antegrade manner, the proximate side port 88 is sometimes referred to as a second side port and the distal port 90 is sometimes referred to as an infusate side port. However, when the access tube 82 advances in an retrograde manner, the proximate side port 88 can be referred to as an infusate side port and the distal port 90 can be referred to as a second side port. It should be understood, however, that if the access tube 82 extends into the vasculature in a retrograde manner, the preferred configuration would be to project the probe 84 from the distal port 90 such that fluid may be infused from the proximal side port 88 without interfering with measurements of the sensor 86.

FIG. 5 illustrates an alternative sensing system 100 comprising an elongated access tube 102 and a sensing probe 104 having a distal sensor 106 thereon. The illustrated access tube 102, which may be a conventional CV catheter, includes multiple lumens, not shown, one each of which lead to a proximal side port 108 and a distal side port 110. While the distal side port 110 may be used to infuse fluid F, the probe 104 extends through the lumen associated with the proximal side port 108 and projects therefrom. To do so, the probe 104 forms two bends, a first bend 112 located inside of the access tube 102 and a second bend 114, to form an S-shape segment adjacent its distal end, thus causing the distal segment to extend laterally from the side port 108. The proximal end of the probe 104 could be provided with a marker (not shown) for rotational orientation to ensure that the bends 112, 114 point toward the side port 108. Furthermore, the probe 104 desirably possesses a minimum torque strength so that it can be twisted if necessary until the distal tip thereof exits the side port 108. By projecting the probe 104 from the proximal side port 108, it remains at all times proximally located with respect to the distal side port 110 and infusate flow F, at least when the access tube 102 advances in an antegrade manner. It should be understood, however, that if the access tube 102 extends into the vasculature in a retrograde manner, the preferred configuration would be to project the probe 104 from the distal port 110 such that fluid may be infused from the proximal side port 108 without interfering with measurements of the sensor 106.

The preceding S-shaped bends 112, 114 result in a relatively linear profile once the probe 104 has exited the access tube 102. Such an arrangement might be well-suited for expulsion from side ports of catheters in large vessels, such as a CV catheter, and alternatively for expulsion from the distal end of catheters in smaller vessels, such as a peripheral line. Moreover, the particular curve formed by the bends 112, 114 is variable, and may even be 3-dimensional such as in a corkscrew shape.

The aforementioned bends in the various probes described above may cause some difficulty when inserting the probe tips into a lumen of a catheter, and may provide resistance when sliding the probe through the catheter lumen. Such issues may be mitigated by incorporating something like a guidewire insertion device, which is to say a lubricious sheath around the probe. If such a sheath is used, it is desirably seamed so as to come apart or tear apart into two or more sections to facilitate separation from and egress of the probe tip.

Furthermore, the systems may be provided with a Touhey-Borst fitting or the like to enable the linearly movable probe to be fixed at a particular location. This way, the probe can be inserted to different depths depending on the catheter in which it is being used. Also, the probe could have markings along its length to aid insertion to the desired depth. If the probe is highly flexible, a proximal insertion device could be utilized which protects the external portions of the probe. For example, a clip that allows the uninserted portion of the probe to form a loop and keeps the probe from flopping around might be used.

Another option that could be incorporated into the systems of the present invention is to permit the probe to be straightened out if desired. For example, the probe could possess an inflation lumen that permits a clinician to inject compressed air or fluid therein to straighten it out during infusion procedures.

Embodiments of the present invention allow the use of a single probe in a variety of axis tubes or catheters. An off-the-shelf probe having the configurable distal end can be coupled with a variety of such catheters, such as various brands of CV catheters.

While the invention has been described in its preferred embodiments, it is to be understood that the words which have been used are words of description and not of limitation. Therefore, changes may be made within the appended claims without departing from the true scope of the invention.

What is claimed is:

1. A medical sensing system, the system comprising:
a vascular access tube comprising:
a distal portion configured for insertion into a vasculature;
a lumen located within said vascular access tube;
an infusate port located at said distal portion of said vascular access tube configured for emitting into the vasculature an infusate input into said lumen during use; and
a sensing probe located in said vascular access tube, said sensing probe comprising a sensor located on a distal portion of said sensing probe, wherein said distal portion of said sensing probe is sized to pass through an access port located at said distal portion of said vascular access tube,
wherein at least one of said distal portions of said vascular access tube and said sensing probe are configured to substantially eliminate communication between said sensor and infusate emitted from said infusate port of said vascular access tube during use.

2. The system of claim 1, wherein said infusate port and said access port are the same port in said vascular access tube.

3. The system of claim 1, wherein said vascular access tube comprises:
a first lumen extending therein connected to said infusate port; and
a second lumen extending therein connected to said access port for receiving said sensing probe.

4. The system of claim 1, wherein said distal portion of said sensing probe is configured for extending from said access port in said vascular access tube so as to orient the sensor at a location outside of a stream of infusate emitted from said infusate port during use.

5. The system of claim 1, wherein said vascular access tube orients said infusate and access ports such that when said distal portion of said sensing probe is configured to extend from said access port in said vascular access tube, said sensor is at a location outside of a stream of infusate from said infusate port.

6. The system of claim 1, wherein said vascular access tube is configured such that said infusate port is located more proximal to said distal end of said vascular access tube than said access port.

7. The system of claim 6, wherein said vascular access tube is configured such that said infusate port is located at a distal end of said vascular access tube and said access port is located at a spaced apart distance from said infusate port on said vascular access tube on a side of said vascular access tube.

8. The system of claim 6, wherein said distal portion of said sensing probe configured to extend from said access port in said vascular access tube is configured to project laterally along a length of the side of said vascular access tube toward said distal end of said vascular access tube.

9. The system of claim 6, wherein said distal portion of said sensing probe configured to extend from said access port in said vascular access tube and is configured to project laterally along a length of the side of said vascular access tube toward said distal end of said vascular access tube such that said sensor is located between said access port and said infusate port.

10. The system of claim 6, wherein said distal portion of said sensing probe configured for extending from said access port in said vascular access tube comprises a bend configured to orient the sensor at a location outside of a stream of infusate from said infusate port during use.

11. The system of claim 10, wherein said bend in said distal portion of said sensing probe creates a curl in the distal portion of said sensing probe, where the curl is configured to orient said sensor upstream of said access port and along a side of said vascular access tube.

12. The system of claim 10, wherein said distal portion of said sensing probe configured for extending from said access port in said vascular access tube comprises first and second bends configure to orient the sensor at a desired location in the vasculature relative to said infusate port.

13. The system of claim 12, wherein the first and second bends form an S-shaped segment in said sensing probe.

14. The system of claim 1, wherein said sensing probe possesses a minimum torque strength so that said sensing probe can be twisted until said distal end thereof exits said access port in said vascular access tube.

15. The system of claim 1, wherein said vascular access tube is configured such that said access port is located more proximal to said distal end of said vascular access tube than said infusate port.

16. The system of claim 15, wherein said vascular access tube is configured such that said access port is located at a distal end of said vascular access tube and said infusate port is located at a spaced apart distance from said access port on said vascular access tube on a side of said vascular access tube.

17. The system of claim 15, wherein said distal portion of said sensing probe configured to extend from said access port in said vascular access tube comprises a bend portion spaced apart from a distal end of said sensing probe, wherein the bend portion is configured to orient said sensor at a location outside of a stream of infusate emitted from said infusate port during use.

18. The system of claim 17, wherein said bend in said distal portion of said sensing probe creates a curl in the distal portion of said sensing probe, where the curl is configured to orient said sensor at a location outside of a stream of infusate emitted from said infusate port during use.

19. The system of claim 15, wherein said distal portion of said sensing probe configured for extending from said access port in said vascular access tube comprises first and second bends configured so as to orient the sensor at a location outside of a stream of infusate emitted from said infusate port during use.

20. The system of claim 19, wherein the first and second bends form an S-shaped segment in said sensing probe.

21. The system of claim 15, wherein said distal portion of said sensing probe configured to extend from said access port in said vascular access tube comprises a bend portion spaced apart from a distal end of said sensing probe, wherein the bend portion is configured to orient said sensor at a location upstream of said infusate port during use.

22. The system of claim 21, wherein said distal portion of said sensing probe comprising said sensor is configured to extend laterally along a length of the side of said vascular access tube during use.

23. The system of claim 17, wherein bend portion of the sensing probe bends at an angle of approximately 180 degrees.

24. The system of claim 18, wherein the curl bends between approximately 180 degrees and approximately 270 degrees.

25. An apparatus, comprising:
an access tube having an infusate port and a second port and a distal end;
a probe partially disposed in said access tube and having a distal portion projecting from the second port of said access tube; and
a sensor disposed on the distal portion of said probe,
wherein said probe bends after projecting from the second port in said access tube so as to orient said sensor at a location outside of a stream of infusate emitted from the infusate port during use.

26. The apparatus of claim 25, wherein the infusate port and the second port are the same port in said access tube.

27. The apparatus of claim 25, wherein said access tube is configured such that the infusate port is located more proximal to the distal end of said access tube than the second port.

28. The apparatus of claim 25, wherein said access tube is configured such that the second port is located more proximal to the distal end of said access tube than the infusate port.

29. The apparatus of claim 25, wherein said access tube is configured such that the second port is located at a distal end of said access tube and the infusate port is located at a spaced apart distance from the second port on said access tube on a side of said access tube.

30. The apparatus of claim 25, wherein at a location where said probe bends said probe is constructed from a resilient material that is elastically-biased.

31. The apparatus of claim 25, wherein at a location where said probe bends said probe is constructed of Nitinol.

32. The apparatus of claim 25, wherein at a location where said probe bends said probe is constructed of a polymer material with a segment of Nitinol wire embedded therein.

33. A method of sensing a blood parameter, comprising:
providing a vascular access tube including a fluid infusate lumen and associated fluid infusate port in a sidewall thereof, the access tube further including a second lumen leading to a second port in the access tube;
providing a sensing probe having a sensor on a distal end thereof, the sensing probe being sized to pass through the second lumen and having a shape at its distal end that permits the distal end to extend from the second port such that the sensor is displaced laterally from the access tube;
introducing the vascular access tube into the vasculature of a patient;
passing the sensing probe through the second lumen of the access tube until the distal end extends from the second port; and
sensing a blood parameter with the sensor while infusing fluid through the fluid infusate lumen and fluid infusate port into the vasculature.
